(19) United States
(12) Patent Application Publication
(10) Pub. No.: US 2026/0325828 A1
(43) Pub. Date: Oct. 22, 2026

(54) Low-Profile Electrical Connectors for Recharging of an Implanted Medical System

(71) Applicant: TC1 LLC, St. Paul, MN (US)

(72) Inventors: John Hai Nguyen, San Ramon, CA (US); Michael Morado, Escalon, CA (US); Daniel I. Harjes, Carlisle, MA (US); Fabian Frigon Franco, Livermore, CA (US); Karen Masterson, Placerville, CA (US); Joseph P. Sylvester, JR., Andover, MA (US); Jeff Iudice, Lowell, MA (US); Eric T. Lee, Oakland, CA (US); Bala Sivaraman, St. Paul, MN (US); Li Jin, St. Paul, MN (US)

(73) Assignee: TC1 LLC, St. Paul, MN (US)

(21) Appl. No.: 19/700,534

(22) Filed: Jun. 8, 2026

Related U.S. Application Data
(63) Continuation of application No. PCT/US2024/058112, filed on Dec. 2, 2024.
(60) Provisional application No. 63/609,469, filed on Dec. 13, 2023.

(51) Int. Cl. A61M 60/875; A61M 60/178; A61M 60/876

(52) U.S. Cl. CPC A61M 60/875; A61M 60/178; A61M 60/876; A61M 2205/0272

(57) ABSTRACT

A mechanical circulatory assist system includes a percutaneous electrical cable with a low-profile electrical connector assembly. A mechanical circulatory assist system includes a ventricular assist device, an implantable battery and a low-profile electrical connector assembly. The implantable battery is configured for supplying power for operation of the ventricular assist device while an external source of electrical power is not connected via the low-profile electrical connector assembly. The low-profile electrical connector includes an implantable electrical connector and an exterior electrical connector that can be selectively connected and disconnected from the implantable electrical connector. The implantable electrical connector can have a low profile relative to a region of skin to reduce the chance of snagging and/or tugging of the implantable electrical connector. Likewise, the external electrical connector can have a low profile relative to the region of skin to reduce the chance of snagging and/or tugging of the external electrical connector.

### CROSS-REFERENCE TO RELATED APPLICATIONS

The present application is a Continuation of PCT/US2024/058112 filed Dec. 2, 2024; which claims the benefit of U.S. Provisional Appln. No. 63/609,469 filed Dec. 13, 2023, the full disclosures which are incorporated herein by reference in their entirety for all purposes.

### BACKGROUND

Ventricular assist devices, known as VADs, are used for both short-term (i.e., days, months) and long-term blood circulation assistance (i.e., years or a lifetime) where a patient's heart is incapable of providing adequate circulation, commonly referred to as heart failure or congestive heart failure. According to the American Heart Association, more than five million Americans are living with heart failure, with about 670,000 new cases diagnosed every year. People with heart failure often have shortness of breath and fatigue. Years of living with blocked arteries and/or high blood pressure can leave a heart too weak to pump enough blood to the body. As symptoms worsen, advanced heart failure develops.

A patient suffering from heart failure may use a VAD while awaiting a heart transplant or as a long term destination therapy. A patient may also use a VAD while recovering from heart surgery. Thus, a VAD can supplement a weak heart (i.e., partial support) or can effectively replace the natural heart's function.

### BRIEF SUMMARY

The following presents a simplified summary of some systems of the present disclosure in order to provide a basic understanding of the systems and methods of the present disclosure. This summary is not an extensive overview of the systems and methods of the present disclosure. It is not intended to identify key/critical elements of the systems and methods of the present disclosure or to delineate the scope of the systems and methods of the present disclosure. Its sole purpose is to present some systems of the present disclosure in a simplified form as a prelude to the more detailed description that is presented later.

Some of the systems described herein are directed to mechanical circulatory assist systems that include a ventricular assist device, an implanted controller that includes a rechargeable battery, and an electrical connector assembly. The electrical connector assembly includes an implantable electrical connector and an external electrical connector. The implanted control unit provides power for operation of the ventricular assist device while the implanted electrical connector is not connected to an external power source via the external electrical connector. Optionally, the implanted electrical connector can include electrical contacts that are disposed on or below the skin surface of the patient to accommodate the usage of an implanted electrical cable (which is used to transmit energy to the implanted control unit from the implanted electrical connector) without an externally exposed segment, which can be uncomfortable, inconvenient, bulky, and prone to tugging and snagging. Optionally, the external electrical connector can be configured to disconnect from the implantable electrical connector when subjected to tugging or snagging to prevent excessive force transfer to the implantable electrical connector. The electrical connector assembly can be configured to reduce the risk of infection and other adverse events due to tugging and snagging due to the electrical connector assembly's low profile configuration.

Thus, in one aspect, a first mechanical circulatory assist system includes a ventricular assist device, an implantable battery, and an electrical connector assembly. The ventricular assist device is configured for pumping blood from a ventricle of a patient to an artery of the patient. The implantable battery is configured for supplying power for operation of the ventricular assist device. The electrical connector assembly includes an implantable electrical connector and an external electrical connector. The implantable electrical connector is configured for receiving electrical power for recharging of the implantable battery. The implantable electrical connector includes implantable connector electrical contacts and an implantable support base to which the implantable connector electrical contacts are mounted and distributed along the implantable support base. The implantable electrical connector is configured to be implanted within the patient so that each of the implantable connector electrical contacts extends from the implantable support base disposed within the patient through a respective aperture in a region of skin of the patient. The external electrical connector includes external connector electrical contacts and an external support base to which the external connector electrical contacts are mounted and distributed along the external support base. The external electrical connector is configured to be connected to the implantable electrical connector so that the external connector electrical contacts are in contact with the implantable connector electrical contacts.

The electrical connector assembly of the first mechanical circulatory assist system can include any suitable number of electrical contacts. For example, a first pair of the implantable connector electrical contacts can be configured for receiving electrical power for recharging of the implantable battery; a second pair of the implantable connector electrical contacts can be configured for receiving electrical power for recharging of the implantable battery to provide redundancy relative to the first pair of the implantable connector electrical contacts; and the implantable connector electrical contacts can include a first communication contact and a second communication contact to provide redundancy relative to the first communication contact.

The implantable connector electrical contacts of the first mechanical circulatory assist system can have any suitable configuration. For example, each of the implantable connector electrical contacts can be spaced apart from adjacent of the implantable connector electrical contacts by at least 3 mm. Each of the implantable connector electrical contacts may be configured to protrude less than a suitable maximum distance from the region of skin of the patient.

The electrical connector assembly of the first mechanical circulatory assist system can include coupling features configured to decouple to limit the magnitude of force that can be transferred to the implantable electrical connector from the external electrical connector. For example, the electrical connector assembly can include one or more magnets configured to align the external electrical connector with the implantable electrical connector and retain the external electrical connector to the implantable electrical connector while accommodating decoupling of the external electrical connector from the implantable electrical connector via a decoupling force of not more than a suitable maximum decoupling force such as, for example 1.0 lbs.

The implantable electrical connector of the first mechanical circulatory assist system can be configured with one or more anchoring features for anchoring the implantable electrical connector within the patient. For example, the implantable support base can include one or more suture apertures for anchoring of the implantable support base within the patient.

The implantable electrical connector of the first mechanical circulatory assist system can be configured for tissue ingrowth to enhance anchoring of the implantable electrical connector within the patient. For example, the implantable support base can be configured for tissue ingrowth for anchoring of the implantable support base within the patient.

Thus, in another aspect, a second mechanical circulatory assist system includes a ventricular assist device, an implantable battery, and an electrical connector assembly. The ventricular assist device is configured for pumping blood from a ventricle of a patient to an artery of the patient. The implantable battery is configured for supplying power for operation of the ventricular assist device. The electrical connector assembly includes an implantable female electrical connector and an external male electrical connector. The implantable female electrical connector is configured for receiving electrical power for recharging of the implantable battery. The external male electrical connector includes an elongated electrical contact support member and annular electrical contacts mounted to and spaced apart along the elongated electrical contact support member. The implantable female electrical connector includes a female connector body having an elongated receptacle and female contact assemblies disposed in and distributed along the elongated receptacle for interfacing with the annular electrical contacts of the external male electrical connector. The implantable female electrical connector is configured to be implanted within the patient so that the female connector body extends from within the patient through an aperture in a region of skin of the patient.

The electrical connector assembly of the second mechanical circulatory assist system can include any suitable number of electrical contacts. For example, a first pair of the female contact assemblies can configured for receiving electrical power for recharging of the implantable battery; a second pair of the female contact assemblies can be configured for receiving electrical power for recharging of the implantable battery to provide redundancy relative to the first pair of the female contact assemblies; and the female contact assemblies can include a first communication contact and a second communication contact to provide redundancy relative to the first communication contact.

The electrical connector assembly of the second mechanical circulatory assist system can be configured to vent fluid during insertion of the external male electrical connector into the implantable female electrical connector. For example, the implantable female electrical connector can include a fluid pathway for venting fluid displaced from the elongated receptacle via insertion of the external male electrical connector into the elongated receptacle. The fluid pathway can be configured to output the fluid external to the patient.

The implantable female electrical connector of the second mechanical circulatory assist system can be configured to inhibit movement of the implantable female electrical connector relative to surrounding tissue of the patient. For example, the female connector body can include radially distributed recesses shaped to interface with a tissue of the patient to inhibit twisting and axial displacement of the implantable female electrical connector relative to the tissue of the patient. The female connector body can include suture apertures for anchoring of the implantable female electrical connector within the patient. The female connector body can be configured for tissue ingrowth for anchoring of the implantable female electrical connector within the patient.

The implantable female electrical connector of the second mechanical circulatory assist system can be configured to inhibit fluid ingression. For example, the implantable female electrical connector can include wiper seal assemblies. Each of the female contact assemblies can be disposed between two of the wiper seal assemblies. Each of the wiper seal assemblies can include an annular housing and an annular seal supported by the annular housing. The annular seal can be configured to sealing engage the external male electrical connector. Each wiper seal assembly can be configured to cooperate with the external male electrical connector to block passage of fluid along the external male electrical connector.

Thus, in another aspect, a third mechanical circulatory assist system includes a ventricular assist device, an implantable battery, and an electrical connector assembly. The ventricular assist device is configured for pumping blood from a ventricle of a patient to an artery of the patient. The implantable battery is configured for supplying power for operation of the ventricular assist device. The electrical connector assembly includes an implantable subdermal electrical connector and an external hypodermic electrical connector. The implantable subdermal electrical connector is configured for receiving electrical power from the external hypodermic electrical connector for recharging of the implantable battery. The implantable subdermal electrical connector is configured to be implanted within the patient under a region of skin of the patient. The external hypodermic electrical connector is configured to be penetrated through the region of skin of the patient to connect the external hypodermic electrical connector to the implantable subdermal electrical connector.

The electrical connector assembly of the third mechanical circulatory assist system can include any suitable number of electrical contacts. For example, the implantable subdermal electrical connector can include subdermal electrical connector contacts; a first pair of the subdermal electrical connector contacts can be configured for receiving electrical power for recharging of the implantable battery; a second pair of the subdermal electrical connector contacts can be configured for receiving electrical power for recharging of the implantable battery to provide redundancy relative to the first pair of the subdermal electrical connector contacts; the subdermal electrical connector contacts can include a first communication contact; and the subdermal electrical connector contacts can include a second communication contact to provide redundancy relative to the first communication contact.

The external hypodermic electrical connector of the third mechanical circulatory assist system can have any suitable configuration. For example, the external hypodermic electrical connector can include an elongated electrical contact support member and annular electrical contacts mounted to and spaced apart along the elongated electrical contact support member. The implantable subdermal electrical connector can include a subdermal connector body having an elongated receptacle and female contact assemblies disposed in and distributed along the elongated receptacle for interfacing with the annular electrical contacts of the external hypodermic electrical connector.

The implantable subdermal electrical connector of the third mechanical circulatory assist system can be configured to inhibit fluid ingression. For example, the implantable subdermal electrical connector can include wiper seal assemblies; each of the female contact assemblies can be disposed between two of the wiper seal assemblies; each of the wiper seal assemblies can include an annular housing and an annular seal supported by the annular housing; each of the annular seals can be configured to sealing engage the external hypodermic electrical connector; and each of the wiper seal assemblies can be configured to cooperate with the external hypodermic electrical connector to block passage of fluid along the external hypodermic electrical connector.

The implantable subdermal electrical connector of the second mechanical circulatory assist system can be configured to inhibit movement of the implantable subdermal electrical connector relative to surrounding tissue of the patient. For example, the subdermal connector body can include radially distributed recesses shaped to interface with a tissue of the patient to inhibit twisting and axial displacement of the implantable subdermal electrical connector relative to the tissue of the patient. The subdermal connector body can include suture apertures for anchoring of the implantable subdermal electrical connector within the patient. The subdermal connector body can be configured for tissue ingrowth for anchoring of the implantable subdermal electrical connector within the patient.

For a fuller understanding of the nature and advantages of the systems and methods of the present disclosure, reference should be made to the ensuing detailed description and accompanying drawings.

### BRIEF DESCRIPTION OF THE DRAWINGS

FIG. 1 illustrates a mechanical circulatory assist system that includes an external peripheral connected via a low-profile electrical connector assembly, in accordance with systems and methods of the present disclosure.

FIG. 2 illustrates the mechanical circulatory assist system of FIG. 1 with the external peripheral disconnected.

FIG. 3 schematically illustrates low-profile electrical contacts of an implantable electrical connector assembly that can be employed in low profile electrical connector assembly of FIG. 1, in accordance with systems and methods of the present disclosure.

FIG. 4 and FIG. 5 illustrate a low-profile electrical connector assembly that can be used in the mechanical circulatory assist system of FIG. 1 and includes an implantable electrical connector with low-profile surface electrical contacts that protrude through the skin of the patient, in accordance with systems and methods of the present disclosure.

FIG. 6 illustrates the use of tunneling adapters for implanting the implantable electrical connector illustrated in FIG. 4 and FIG. 5.

FIG. 7 illustrates another low-profile electrical connector assembly that can be used in the mechanical circulatory assist system of FIG. 1 and includes an implantable female electrical connector and an external male electrical connector, in accordance with systems and methods of the present disclosure.

FIG. 8 shows a cross-sectional view of the implantable female electrical connector of FIG. 7 coupled with the external male electrical connector.

FIG. 9 illustrates the implantable female electrical connector of FIG. 7 and the external male electrical connector disconnected from the implantable female electrical connector.

FIG. 10 illustrates the implantable female electrical connector of FIG. 7.

FIG. 11 shows a cross-sectional view of the implantable female electrical connector of FIG. 7.

FIG. 12 and FIG. 13 show pictures of a prototype of the low-profile electrical connector assembly of FIG. 7.

FIG. 14 illustrates another low-profile electrical connector assembly that can be used in the mechanical circulatory assist system of FIG. 1 and includes an implantable subdermal electrical connector and an external hypodermic electrical connector, in accordance with systems and methods of the present disclosure.

FIG. 15 and FIG. 16 illustrate an example configuration of the low-profile electrical connector assembly of FIG. 14 with a configuration similar to the low-profile electrical connector of FIG. 7, in accordance with systems and methods of the present disclosure.

### DETAILED DESCRIPTION

In the following description, various systems and methods of the present disclosure will be described. For purposes of explanation, specific configurations and details are set forth in order to provide a thorough understanding of the described systems and methods. However, it will also be apparent to one skilled in the art that the systems and methods of the present disclosure may be practiced without the specific details. Furthermore, well-known features may be omitted or simplified in order not to obscure the systems and methods being described.

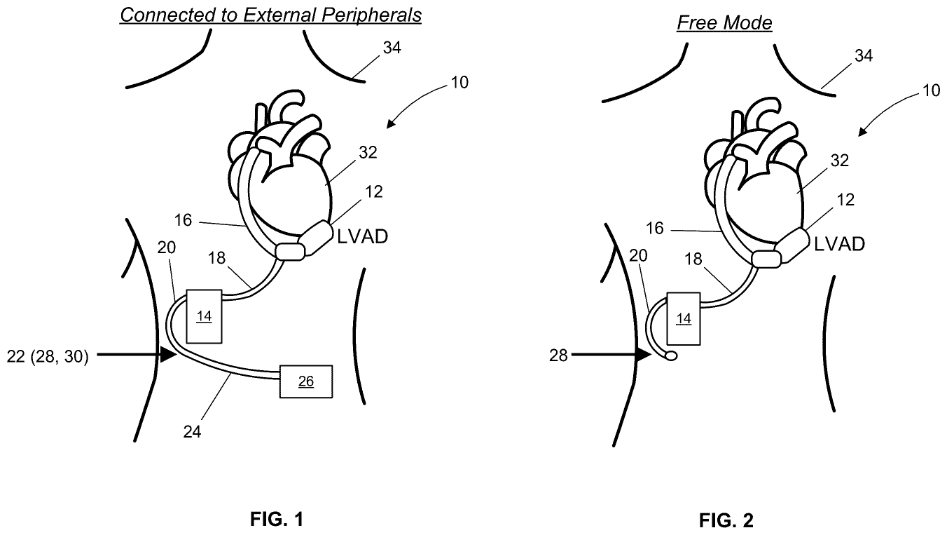

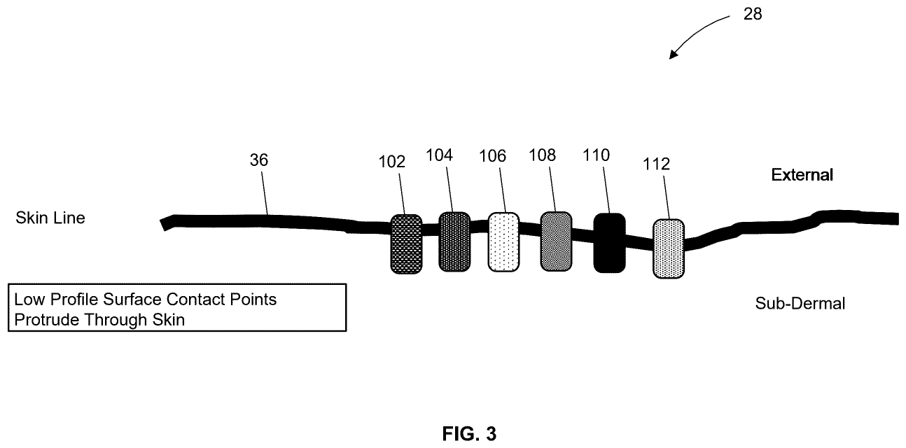

Turning now to the drawing figures in which similar reference identifiers refer to similar features, FIG. 1 illustrates a mechanical circulatory assist system 10 in accordance with systems and methods of the present disclosure. The mechanical circulatory assist system 10 includes a ventricular assist device (VAD) 12, an implantable controller 14, an outflow conduit 16, a first connection cable 18, and an implanted second connection cable 20, a low-profile electrical connector assembly 22, an external electrical cable 24, and an external peripheral unit 26. The low-profile electrical connector assembly 22 includes an implantable electrical connector 28 and an external electrical connector 30 configured for connection to the implantable electrical connector 28. The VAD 12 is operable to pump blood from a ventricle of the heart 32 (e.g., left ventricle, right ventricle) of a patient 34 to an artery (e.g., the aorta when the VAD 12 is employed as a left ventricular assist device, the pulmonary artery when the VAD 12 is employed as a right ventricular assist device) to supplement pumping of blood by the ventricle to the artery. The implantable controller 14 can be configured to control operation of the VAD 12. The implantable controller 14 can include a rechargeable implanted battery configured to provide electrical power for operation of the VAD 12 and the controller 14 in the system configuration illustrated in FIG. 2 in which the external peripheral unit 26 is disconnected from the implantable controller 14. The rechargeable implanted battery, however, can be separate from the implantable controller 14 and separately implanted in the patient 34. Due the amount of power required, operation of the VAD 12 in the system configuration illustrated in FIG. 2 may discharge the rechargeable implanted battery at a rate that may require occasional recharging of the rechargeable implanted battery using the system configuration illustrated in FIG. 1. The external peripheral unit 26 can have any suitable configuration in which the external peripheral unit 26 is configured to supply power for simultaneous operation of the VAD 12 and the controller 14 and recharging of the implanted battery. For example, the external peripheral unit 26 can be, for example, an external control unit that includes a rechargeable battery configured to supply power for operation of the VAD 12 and the controller 14 and recharging of the implanted battery. As another example, the external peripheral unit 26 can be an external power supply powered by any suitable electrical power (e.g., alternating current electrical power supplied by a utility power source). As another example, the external peripheral unit 26 can include one or more external rechargeable batteries configured to supply power for operation of the VAD 12 and the controller 14 and recharging of the implanted battery.

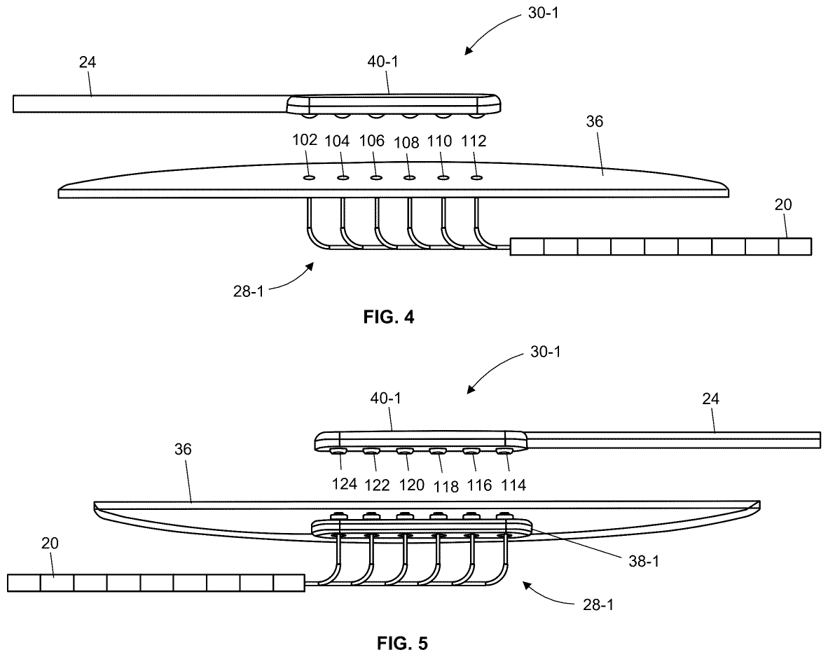

FIG. 3 schematically illustrates low-profile electrical contacts 102, 104, 106, 108, 110, 112 that can be employed in the implantable electrical connector 28, in accordance with systems and methods of the present disclosure. Each of the electrical contacts 102, 104, 106, 108, 110, 112 can extend through a respective aperture in the skin 36 of the patient 34. One pair of the electrical contacts is used for power transmission (e.g., direct current, alternating current), a second pair of the electrical contacts provides redundancy for power transmission. One of the remaining two electrical contacts is used for communication. The final remaining electrical contact provides redundancy for communication. Alternatively, the electrical contacts 102, 104, 106, 108, 110, 112 can be mounted to a base support layer that is secured to the external surface of the skin 36 of the patient 34 and the implanted second connection cable 20 extends through a single aperture in the skin 36 disposed adjacent to or underneath the base support layer. Optionally, each of the first and second connection cables 18, 20 can include at least 6 separate conductors-one for each of the electrical contacts 102, 104, 106, 108, 110, 112.

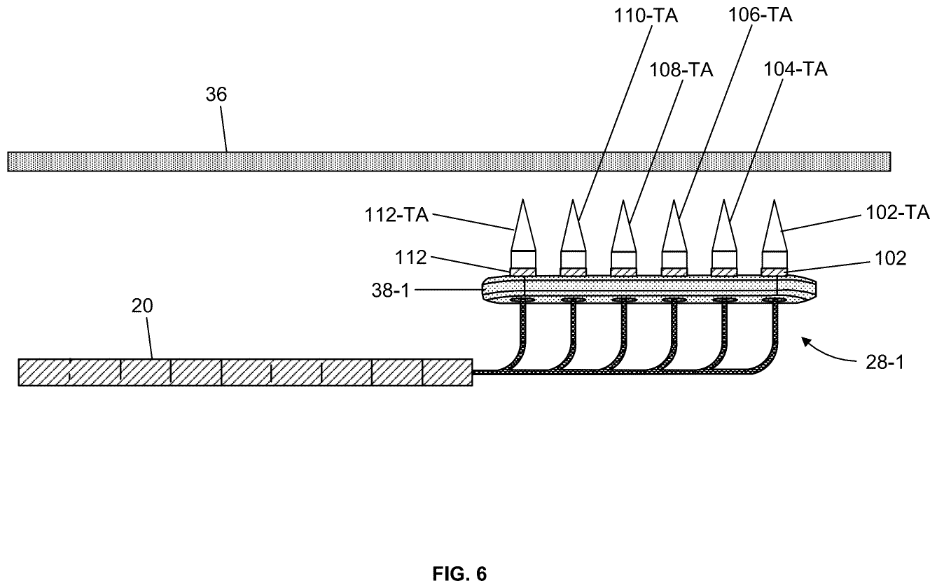

FIG. 4 and FIG. 5 illustrate an example 22-1 of the low-profile electrical connector assembly 22. The example 22-1 includes an example 28-1 of the implanted electrical connector 28 and an example 30-1 of the external electrical connector 30. The implanted electrical connector 28-1 includes a base support layer 38-1 and the electrical contacts 102, 104, 106, 108, 110, 112 mounted to the base support layer 38-1. The external electrical connector 30-1 includes a contact support 40-1 and electrical contacts 114, 116, 118, 120, 122, 124 mounted to the contact support 40-1. The implanted electrical connector 28-1 and the external electrical connector 30-1 can include alignment and retention features (e.g., magnets, complementarily shaped coupling features) configured for alignment and to provide a suitable level of retention of the external electrical connector 30-1 with and to the implantable electrical connector 28-1. When connected, each of the electrical contacts 114, 116, 118, 120, 122, 124 of the external electrical connector 30-1 is held in contact with a corresponding one of the electrical contacts 102, 104, 106, 108, 110, 112 of the implanted electrical connector 28-1 Optionally, the alignment and retention features can be configured to limit the maximum force that can be transferred to the implanted electrical connector 28-1 by the external electrical connector 30-1 via disconnection of the external electrical connector 30-1 from the implantable electrical connector 28-1.

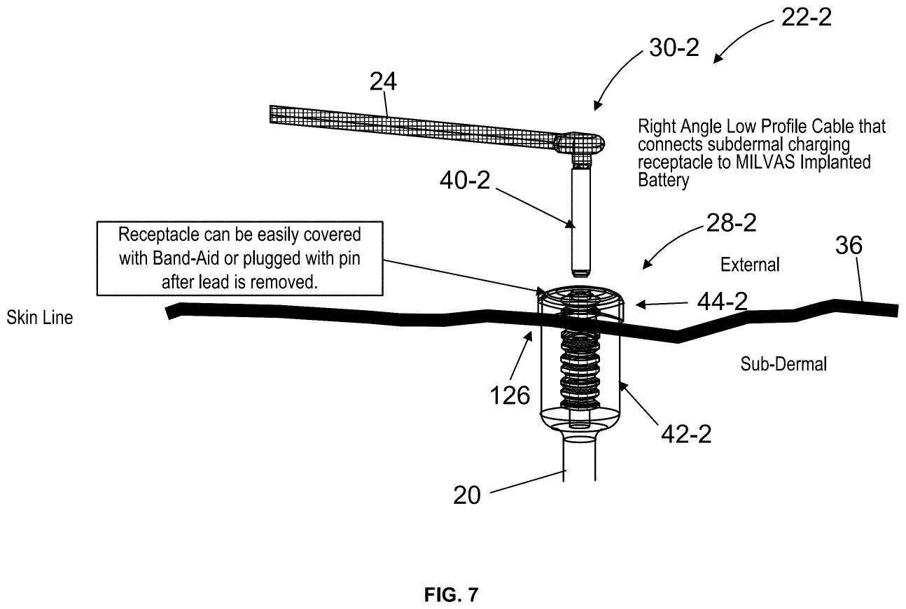

FIG. 6 shows implantation adapters 102-TA, 104-TA, 106-TA, 108-TA, 110-TA, 112-TA configured for forming apertures in the skin 36 for the electrodes 102, 104, 106, 108, 110, 112 of the implantable electrical connector 28-1. The implantation adapters 102-TA, 104-TA, 106-TA, 108-TA, 110-TA, 112-TA are detachably mountable to the electrodes 102, 104, 106, 108, 110, 112 and shaped for penetrating through the skin 36 and guiding of the electrodes 102, 104, 106, 108, 110, 112 into and through the resulting apertures in the skin 36. Following the penetration of the electrodes 102, 104, 106, 108, 110, 112 through the skin 36, the implantation adapters 102-TA, 104-TA, 106-TA, 108-TA, 110-TA, 112-TA are detached from the electrodes 102, 104, 106, 108, 110, 112. The base support layer 38-1 can include suture holes for suturing of the base support layer 38-1 to a suitable adjacent tissue of the patient 34 to retain the implantable electrical connector 28-1 in a suitable fixed position and orientation relative to the skin 36 following the penetration of the electrodes 102, 104, 106, 108, 110, 112 through the skin 36. Optionally, the base support layer 38-1 can be made from a suitable biocompatible material (e.g., titanium). The base support layer 38-1 and/or the electrodes 102, 104, 106, 108, 110, 112 can be configured for tissue ingrowth. For example, the base support layer 38-1 and/or the electrodes 102, 104, 106, 108, 110, 112 can have a sintered titanium external coating configured to induce tissue ingrowth.

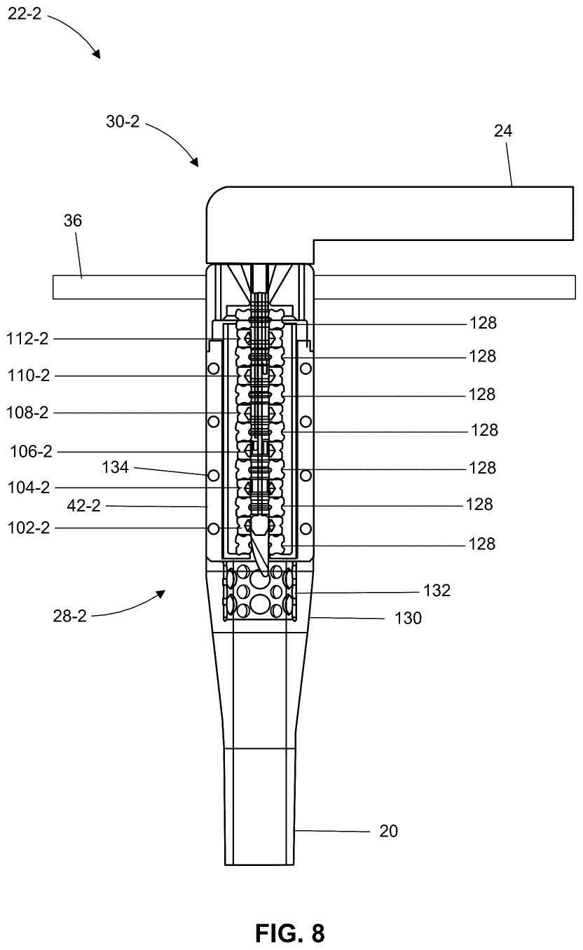

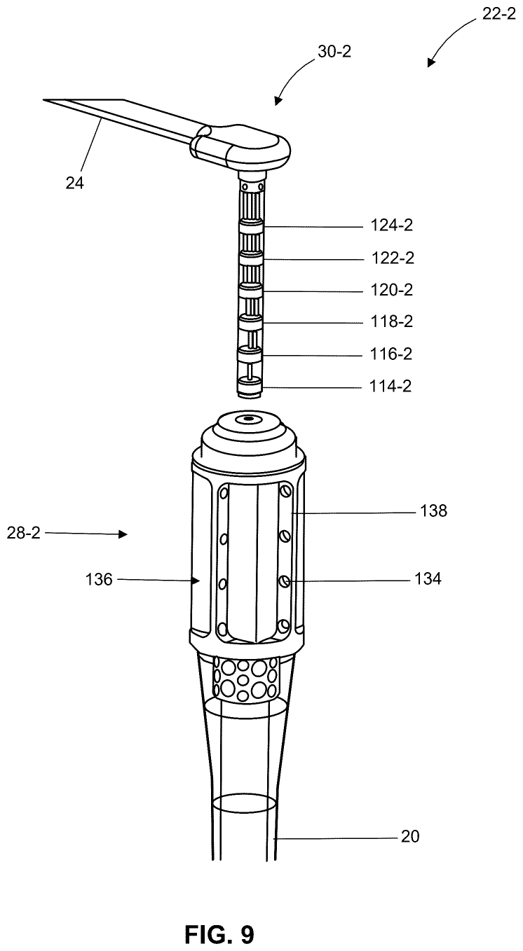

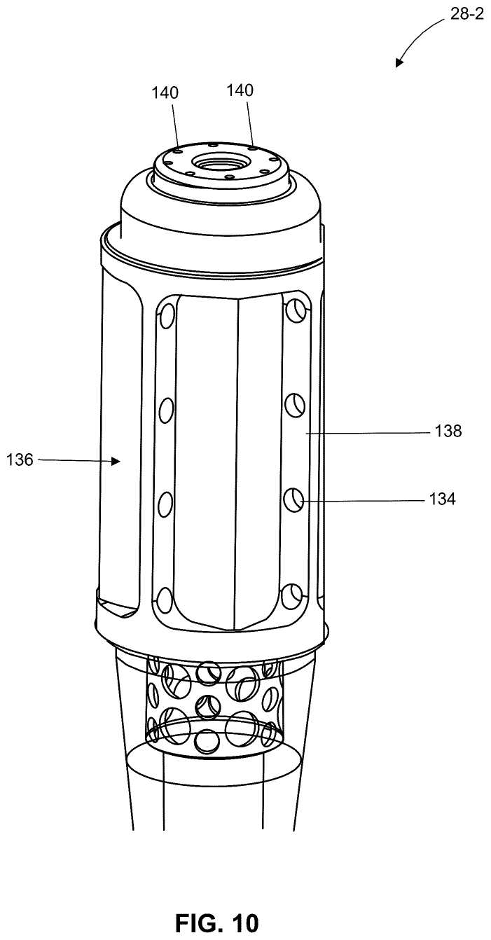

FIG. 7 illustrates another example 22-2 of the low-profile electrical connector assembly 22. The low-profile electrical connector assembly 22-2 includes an example 28-2 of the implanted electrical connector 28 and an example 30-2 of the external electrical connector 30. The implanted electrical connector 28-2 includes a female receptacle 126 with spaced apart electrical contacts 102-2, 104-2, 106-2, 108-2, 110-2, 112-2 (also shown in FIG. 8). The electrical contacts 102-2, 104-2, 106-2, 108-2, 110-2, 112-2 can have the same functional attributes as the electrical contacts 102, 104, 106, 108, 110, 112 for transmission of power, redundancy of power transmission, communication, and redundancy of communication. The external electrical connector 30-2 includes a male lead 40-2 with spaced apart electrical contacts 114-2, 116-2, 118-2, 120-2, 122-2, 124-2 (shown in FIG. 9). The implanted electrical connector 28-2 includes a receptacle body 42-2 configured to be anchored under the skin 36 for stability. The electrical connector 28-2 can be implanted so that a proximal end portion 44-2 of the receptacle body 20-2 extends through an aperture in the skin 36. The external electrical connector 30-2 is configured so that the external electrical cable 24 extends perpendicular to the male lead 40-2 so as to position the distal end portion of the external electrical cable 24 adjacent to the skin 36 to reduce likelihood of snagging of the external electrical cable 24. While disconnected from the external electrical connector 30-2, the female receptacle of the implanted electrical connector 28-2 can be covered by a suitable barrier (e.g., a “Band-Aid”, a detachably mountable distal end cap) and/or plugged with a suitable male plug.

FIG. 8 shows a cross-sectional view of the low-profile electrical connector assembly 22-2 with implantable electrical connector 28-2 coupled with the external male electrical connector 30-2. The implantable electrical connector 28-2 further includes wiper seals 128. Each of the spaced apart electrical contacts 114-2, 116-2, 118-2, 120-2, 122-2, 124-2 is sandwiched between two of the wiper seals 128. Each of the wiper seals is configured to engage the male lead 40-2 of the external electrical connector 30-2 to block flow of fluid along the surface of the male lead 40-2. The implantable electrical connector 28-2 further includes a strain relief member 130 configured for constraining the proximal end portion of the implanted second connection cable 20 relative to the receptacle body 42-2 to reduce strains in the proximal end portion of the implanted second connection cable 20 to enhance the durability of the proximal end portion of the implanted second connection cable 20 and the electrical connections between conductors of the implanted second connection cable 20 and the spaced apart electrical contacts 114-2, 116-2, 118-2, 120-2, 122-2, 124-2. The strain relief member 130 can be formed from a suitable biocompatible material and is molded over a molding support feature 132 of the receptacle body 42-2. In the illustrated example, the molding support feature 132 includes a pattern of apertures configured to enhance attachment of the molded strain relief member 130 to the molding support feature 132. The receptacle body 42-2 includes suture holes 134 for use in securing the receptacle body 42-2 to surrounding tissue via sutures.

FIG. 9 shows the low-profile electrical connector assembly 22-2 with the external male electrical connector 30-2 separated from the implantable electrical connector 28-2. The receptacle body 42-2 has an exterior shape configured to secure the receptacle body 42-2 to the surrounding tissue to inhibit changes in position and orientation of the receptacle body 42-2 relative to the surrounding tissue. In the illustrated example, the receptacle body 42-2 defines four circumferentially distributed recesses 136 and four flanges 138, which extend both longitudinally and radially relative to a longitudinal centerline of the receptacle body 42-2. Four of the suture holes 134 extend circumferentially through each of the flanges 138. The receptacle body 42-2 can have an external surface configured to promote tissue ingrowth. For example, the receptacle body 42-2 can be made from a suitable titanium and have a suitable sintered titanium coating configured to promote tissue ingrowth.

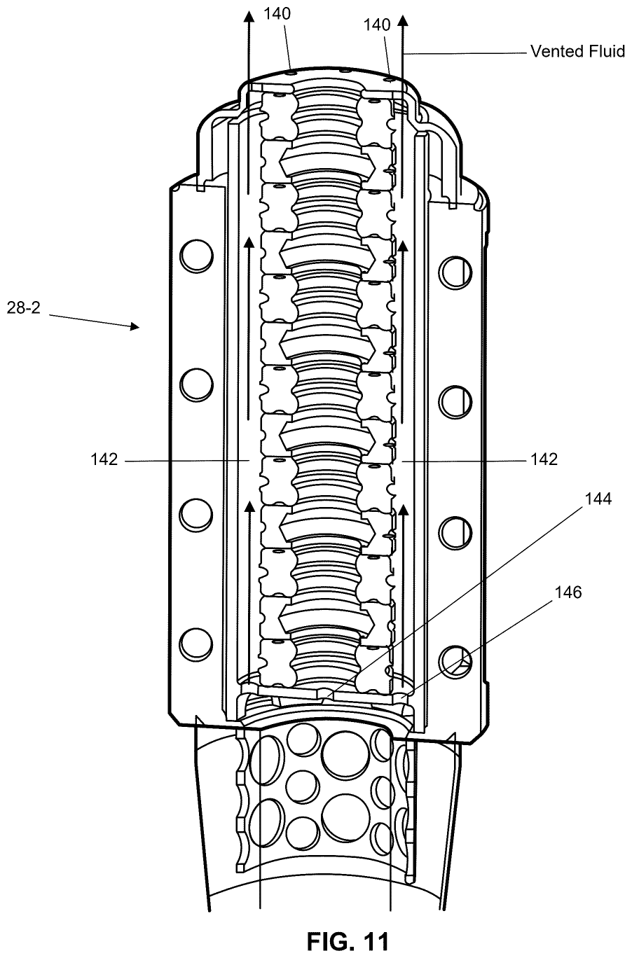

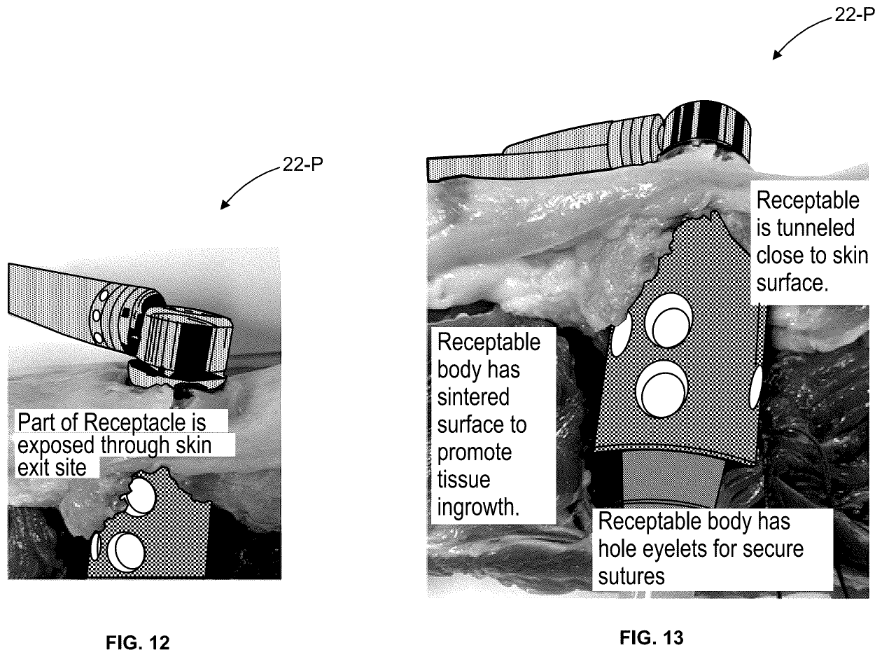

FIG. 10 shows a close-up view of the implantable electrical connector 28-2. The implantable electrical connector 28-2 further includes eight fluid vent holes 140 for venting fluid displaced during insertion of the male lead 40-2 of the external electrical connector 30-2 into the implantable electrical connector 28-2. As shown in the cross-sectional view of FIG. 11, the receptacle body 42-2 defines an annular fluid channel 142 and fluid holes 144, 146 that form a fluid pathway to the vent holes 140 for venting of the fluid displaced during insertion of the male lead 40-2 of the external electrical connector 30-2 into the implantable electrical connector 28-2.

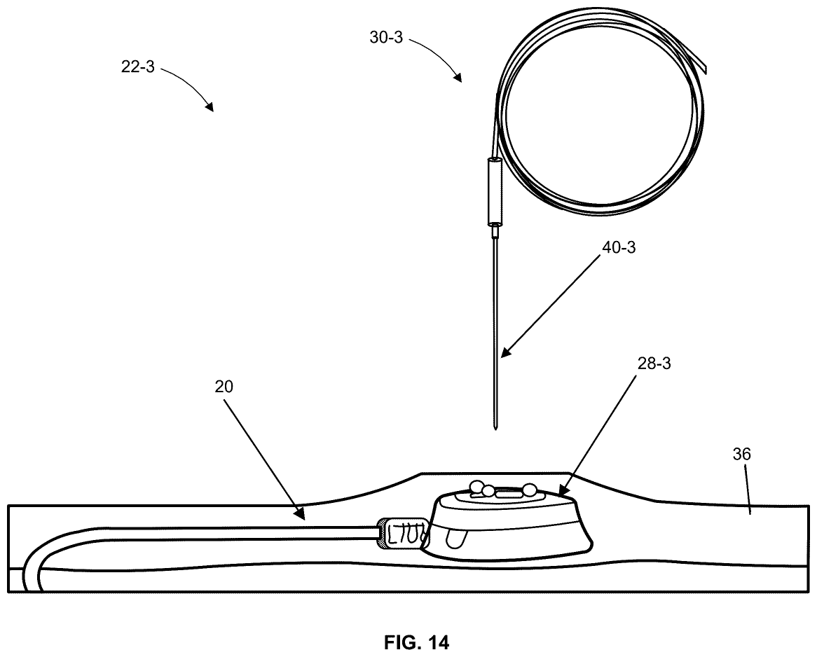

FIG. 12 and FIG. 13 show pictures of an implanted prototype 22-P of the low-profile electrical connector assembly 22. In the implanted configuration shown in FIG. 12, a proximal end part of the prototype implantable female electrical connector 28 is exposed above the skin to accommodate insertion of the prototype external male electrical connector 30. As best seen in FIG. 13, the receptacle body of the prototype implantable female electrical connector 28 has a sintered surface to promote tissue ingrowth and suture holes for securing sutures used to secure the prototype implantable electrical connector 28 to surrounding tissue.

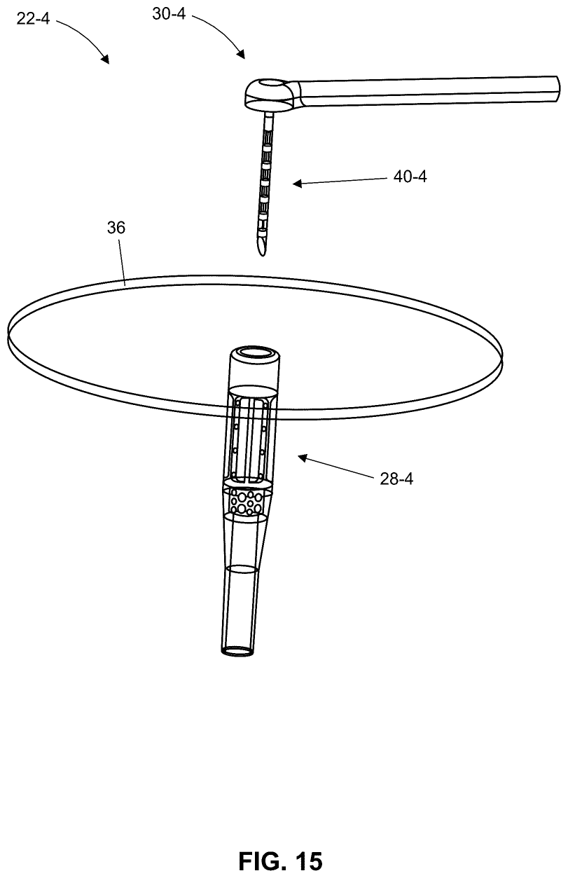

FIG. 14 illustrates another example 22-3 of the low-profile electrical connector assembly 22. The low-profile electrical connector 22-3 includes an implantable subdermal electrical connector 28-3 and an external hypodermic electrical connector 30-3. The subdermal electrical connector 28-3 is configured to be implanted below the exterior surface of the skin 36. The external hypodermic electrical connector 30-3 includes a small diameter (e.g., from 1 mm to 5 mm) elongated contact assembly 40-3 configured to be penetrated through the overlying skin 36 into the subdermal electrical connector 38-3. The elongated contact assembly 40-3 can include any suitable number of spaced apart male electrical contacts (e.g., 1, 2, 3, 4, 5, 6, or more) and the subdermal electrical connector 28-3 can have a corresponding number of spaced apart female electrical contacts. Each of the spaced apart female electrical contacts can be configured to maintain contact with a respective one of the spaced apart male electrical contacts while the electrical connector 22 is in a connected configuration.

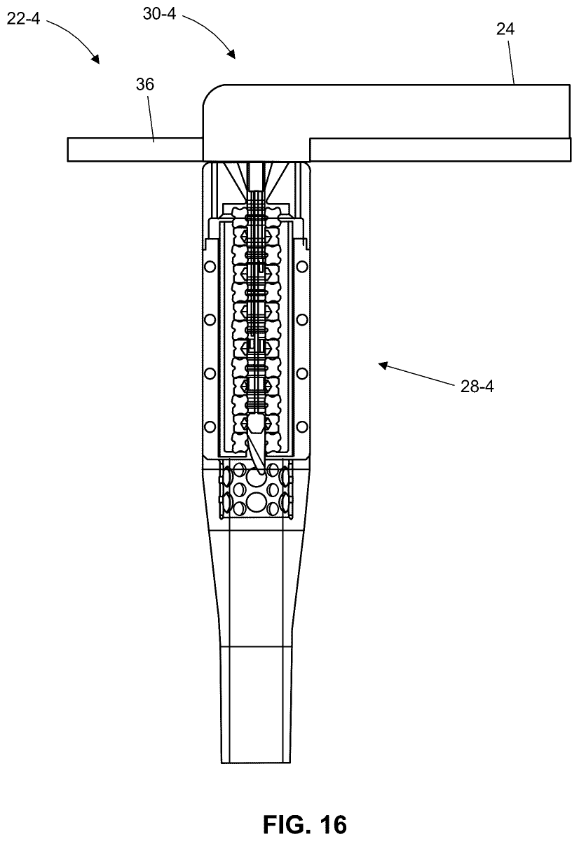

FIG. 15 and FIG. 16 illustrate an example 22-4 of the low-profile electrical connector assembly 22-3. The low-profile electrical connector assembly 22-4 includes an example 30-4 of the external hypodermic electrical connector 30-3 and an example 28-4 of the implantable subdermal electrical connector 28-3. The low-profile electrical connector assembly 22-4 is configured the same as the low-profile electrical connector 22-2 except for the diameter of the elongate contact assembly 40-4 being smaller (e.g., from 1 mm to 6 mm in diameter) than the diameter of the male lead 40-2 (which can be in a range from 1 mm to 5 mm). The implantable subdermal electrical connector 28-4 is configured the same as the implantable electrical connector 28-2 except for having a smaller diameter receptacle for receiving the elongate contact assembly 40-4 and configured to be implanted beneath the external surface of the skin 36. Optionally, the implantable subdermal electrical connector 28-4 can be configured without the provisions for venting of fluid during insertion of the elongate contact assembly 40-4 into the electrical connector 28-2 as the volume of fluid displaced is much reduced due to the smaller receptacle diameter. Except for the above-described differences, the description of the low-profile electrical connector assembly 22-2 is applicable to the low-profile electrical connector assembly 22-4.

Other variations are within the spirit of the systems and methods of the present disclosure. For example, while systems and methods of the present disclosure are described herein in the context of a mechanical circulatory assist system, the low-profile electrical connector assemblies 22, 22-1, 22-2, 22-3, 22-4 can be employed in any suitable implantable medical device system that employs an implantable rechargeable battery. Such suitable implantable medical device systems include, but are not limited to, cardiac rhythm management systems, neurological stimulation systems, and spinal stimulation systems. Thus, while the systems and methods of the present disclosure are susceptible to various modifications and alternative constructions, certain illustrated systems and methods thereof are shown in the drawings and have been described above in detail. It should be understood, however, that there is no intention to limit the systems and methods of the present disclosure to the specific form or forms disclosed, but on the contrary, the intention is to cover all modifications, alternative constructions, and equivalents falling within the spirit and scope of the systems and methods of the present disclosure, as defined in the appended claims.

The use of the terms “a” and “an” and “the” and similar referents in the context of describing the systems and methods of the present disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate systems and methods of the present disclosure and does not pose a limitation on the scope of the systems and methods of the present disclosure unless otherwise claimed. No language in the specification should be construed as indicating any non-claimed element as essential to the practice of the systems and methods of the present disclosure.

Preferred systems and methods of the present disclosure are described herein, including the best mode known to the inventors for carrying out the systems and methods of the present disclosure. Variations of those preferred systems and methods of the present disclosure may become apparent to those of ordinary skill in the art upon reading the foregoing description. The inventors expect skilled artisans to employ such variations as appropriate, and the inventors intend for the systems and methods of the present disclosure to be practiced otherwise than as specifically described herein. Accordingly, the systems and methods of the present disclosure include all modifications and equivalents of the subject matter recited in the claims appended hereto as permitted by applicable law. Moreover, any combination of the above-described elements in all possible variations thereof is encompassed by the systems and methods of the present disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

The following relate to numbered aspects of the invention:
1. A mechanical circulatory assist system comprising:
   a ventricular assist device configured for pumping blood from a ventricle of a patient to an artery of the patient;
   an implantable battery configured for supplying power for operation of the ventricular assist device; and
   an electrical connector assembly comprising an implantable electrical connector and an external electrical connector, wherein the implantable electrical connector is configured for receiving electrical power for recharging of the implantable battery, wherein the implantable electrical connector comprises implantable connector electrical contacts and an implantable support base to which the implantable connector electrical contacts are mounted and distributed along the implantable support base, wherein the implantable electrical connector is configured to be implanted within the patient so that each of the implantable connector electrical contacts extends from the implantable support base disposed within the patient through a respective aperture in a region of skin of the patient, wherein the external electrical connector comprises external connector electrical contacts and an external support base to which the external connector electrical contacts are mounted and distributed along the external support base, and wherein the external electrical connector is configured to be connected to the implantable electrical connector so that the external connector electrical contacts are in contact with the implantable connector electrical contacts.
2. The mechanical circulatory assist system of aspect 1, wherein:
   a first pair of the implantable connector electrical contacts is configured for receiving electrical power for recharging of the implantable battery;
   a second pair of the implantable connector electrical contacts is configured for receiving electrical power for recharging of the implantable battery to provide redundancy relative to the first pair of the implantable connector electrical contacts;
   the implantable connector electrical contacts comprise a first communication contact; and
   the implantable connector electrical contacts comprise a second communication contact to provide redundancy relative to the first communication contact.
3. The mechanical circulatory assist system of aspect 1 or 2, wherein each of the implantable connector electrical contacts is spaced apart from adjacent of the implantable connector electrical contacts by at least 3 mm.
4. The mechanical circulatory assist system of any preceding aspect, wherein the electrical connector assembly comprises one or more magnets configured to align the external electrical connector with the implantable electrical connector and retain the external electrical connector to the implantable electrical connector while accommodating decoupling of the external electrical connector from the implantable electrical connector via a decoupling force of not more than 1.0 lbs.
5. The mechanical circulatory assist system of any preceding aspect, wherein the implantable support base comprises one or more suture apertures for anchoring of the implantable support base within the patient.
6. The mechanical circulatory assist system of any preceding aspect, wherein the implantable support base is configured for tissue ingrowth for anchoring of the implantable support base within the patient.
7. A mechanical circulatory assist system comprising:
   a ventricular assist device configured for pumping blood from a ventricle of a patient to an artery of the patient;
   an implantable battery configured for supplying power for operation of the ventricular assist device; and
   an electrical connector assembly comprising an implantable female electrical connector and an external male electrical connector, wherein the implantable female electrical connector is configured for receiving electrical power for recharging of the implantable battery, wherein the external male electrical connector comprises an elongated electrical contact support member and annular electrical contacts mounted to and spaced apart along the elongated electrical contact support member, wherein the implantable female electrical connector comprises a female connector body having an elongated receptacle and female contact assemblies disposed in and distributed along the elongated receptacle for interfacing with the annular electrical contacts of the external male electrical connector, and wherein the implantable female electrical connector is configured to be implanted within the patient so that the female connector body extends from within the patient through an aperture in a region of skin of the patient.
8. The mechanical circulatory assist system of aspect 7, wherein:
   a first pair of the female contact assemblies is configured for receiving electrical power for recharging of the implantable battery;
   a second pair of the female contact assemblies is configured for receiving electrical power for recharging of the implantable battery to provide redundancy relative to the first pair of the female contact assemblies;
   the female contact assemblies comprise a first communication contact; and the female contact assemblies comprise a second communication contact to provide redundancy relative to the first communication contact.
9. The mechanical circulatory assist system of aspect 7 or 8, wherein:
   the implantable female electrical connector comprises a fluid pathway for venting fluid displaced from the elongated receptacle via insertion of the external male electrical connector into the elongated receptacle; and
   the fluid pathway is configured to output the fluid external to the patient.
10. The mechanical circulatory assist system of any preceding aspect, wherein the female connector body comprises radially distributed recesses shaped to interface with a tissue of the patient to inhibit twisting and axial displacement of the implantable female electrical connector relative to the tissue of the patient.
11. The mechanical circulatory assist system of any preceding aspect, wherein the female connector body comprises suture apertures for anchoring of the implantable female electrical connector within the patient.
12. The mechanical circulatory assist system of any preceding aspect, wherein the female connector body is configured for tissue ingrowth for anchoring of the implantable female electrical connector within the patient.
13. The mechanical circulatory assist system of any preceding aspect, wherein:
   the implantable female electrical connector comprises wiper seal assemblies;
   each of the female contact assemblies is disposed between two of the wiper seal assemblies;
   each of the wiper seal assemblies comprises an annular housing and an annular seal supported by the annular housing;
   the annular seal is configured to sealing engage the external male electrical connector; and
   the wiper seal assembly is configured to cooperate with the external male electrical connector to block passage of fluid along the external male electrical connector.
14. A mechanical circulatory assist system comprising:
   a ventricular assist device configured for pumping blood from a ventricle of a patient to an artery of the patient;
   an implantable battery configured for supplying power for operation of the ventricular assist device; and
   an electrical connector assembly comprising an implantable subdermal electrical connector and an external hypodermic electrical connector, wherein the implantable subdermal electrical connector is configured for receiving electrical power from the external hypodermic electrical connector for recharging of the implantable battery, wherein the implantable subdermal electrical connector is configured to be implanted within the patient under a region of skin of the patient, and wherein the external hypodermic electrical connector is configured to be penetrated through the region of skin of the patient to connect the external hypodermic electrical connector to the implantable subdermal electrical connector.
15. The mechanical circulatory assist system of aspect 14, wherein:
   the implantable subdermal electrical connector comprises subdermal electrical connector contacts;
   a first pair of the subdermal electrical connector contacts is configured for receiving electrical power for recharging of the implantable battery;
   a second pair of the subdermal electrical connector contacts is configured for receiving electrical power for recharging of the implantable battery to provide redundancy relative to the first pair of the subdermal electrical connector contacts;
   the subdermal electrical connector contacts comprise a first communication contact; and
   the subdermal electrical connector contacts comprise a second communication contact to provide redundancy relative to the first communication contact.
16. The mechanical circulatory assist system of aspect 14 or 15, wherein:
   the external hypodermic electrical connector comprises an elongated electrical contact support member and annular electrical contacts mounted to and spaced apart along the elongated electrical contact support member; and
   the implantable subdermal electrical connector comprises a subdermal connector body having an elongated receptacle and female contact assemblies disposed in and distributed along the elongated receptacle for interfacing with the annular electrical contacts of the external hypodermic electrical connector.
17. The mechanical circulatory assist system of aspect 16, wherein:
   the implantable subdermal electrical connector comprises wiper seal assemblies;
   each of the female contact assemblies is disposed between two of the wiper seal assemblies; and
   each of the wiper seal assemblies comprises an annular housing and an annular seal supported by the annular housing, wherein the annular seal is configured to sealing engage the external hypodermic electrical connector, and the wiper seal assembly is configured to cooperate with the external hypodermic electrical connector to block passage of fluid along the external hypodermic electrical connector.
18. The mechanical circulatory assist system of aspect 16 or 17, wherein the subdermal connector body comprises radially distributed recesses shaped to interface with a tissue of the patient to inhibit twisting and axial displacement of the implantable subdermal electrical connector relative to the tissue of the patient.
19. The mechanical circulatory assist system of any one of aspects 16 to 18, wherein the subdermal connector body comprises suture apertures for anchoring of the implantable subdermal electrical connector within the patient.
20. The mechanical circulatory assist system of any one of aspects 16 to 19, wherein the subdermal connector body is configured for tissue ingrowth for anchoring of the implantable subdermal electrical connector within the patient.

What is claimed is:

1. A mechanical circulatory assist system comprising:
a ventricular assist device configured for pumping blood from a ventricle of a patient to an artery of the patient;
an implantable battery configured for supplying power for operation of the ventricular assist device; and
an electrical connector assembly comprising an implantable electrical connector and an external electrical connector, wherein the implantable electrical connector is configured for receiving electrical power for recharging of the implantable battery, wherein the implantable electrical connector comprises implantable connector electrical contacts and an implantable support base to which the implantable connector electrical contacts are mounted and distributed along the implantable support base, wherein the implantable electrical connector is configured to be implanted within the patient so that each of the implantable connector electrical contacts extends from the implantable support base disposed within the patient through a respective aperture in a region of skin of the patient, wherein the external electrical connector comprises external connector electrical contacts and an external support base to which the external connector electrical contacts are mounted and distributed along the external support base, and wherein the external electrical connector is configured to be connected to the implantable electrical connector so that the external connector electrical contacts are in contact with the implantable connector electrical contacts.

2. The mechanical circulatory assist system of claim 1, wherein:
a first pair of the implantable connector electrical contacts is configured for receiving electrical power for recharging of the implantable battery;
a second pair of the implantable connector electrical contacts is configured for receiving electrical power for recharging of the implantable battery to provide redundancy relative to the first pair of the implantable connector electrical contacts;
the implantable connector electrical contacts comprise a first communication contact; and
the implantable connector electrical contacts comprise a second communication contact to provide redundancy relative to the first communication contact.

3. The mechanical circulatory assist system of claim 1, wherein each of the implantable connector electrical contacts is spaced apart from adjacent of the implantable connector electrical contacts by at least 3 mm.

4. The mechanical circulatory assist system of claim 1, wherein the electrical connector assembly comprises one or more magnets configured to align the external electrical connector with the implantable electrical connector and retain the external electrical connector to the implantable electrical connector while accommodating decoupling of the external electrical connector from the implantable electrical connector via a decoupling force of not more than 1.0 lbs.

5. The mechanical circulatory assist system of claim 1, wherein the implantable support base comprises one or more suture apertures for anchoring of the implantable support base within the patient.

6. The mechanical circulatory assist system of claim 1, wherein the implantable support base is configured for tissue ingrowth for anchoring of the implantable support base within the patient.

7. A mechanical circulatory assist system comprising:
a ventricular assist device configured for pumping blood from a ventricle of a patient to an artery of the patient;
an implantable battery configured for supplying power for operation of the ventricular assist device; and
an electrical connector assembly comprising an implantable female electrical connector and an external male electrical connector, wherein the implantable female electrical connector is configured for receiving electrical power for recharging of the implantable battery, wherein the external male electrical connector comprises an elongated electrical contact support member and annular electrical contacts mounted to and spaced apart along the elongated electrical contact support member, wherein the implantable female electrical connector comprises a female connector body having an elongated receptacle and female contact assemblies disposed in and distributed along the elongated receptacle for interfacing with the annular electrical contacts of the external male electrical connector, and wherein the implantable female electrical connector is configured to be implanted within the patient so that the female connector body extends from within the patient through an aperture in a region of skin of the patient.

8. The mechanical circulatory assist system of claim 7, wherein:
a first pair of the female contact assemblies is configured for receiving electrical power for recharging of the implantable battery;
a second pair of the female contact assemblies is configured for receiving electrical power for recharging of the implantable battery to provide redundancy relative to the first pair of the female contact assemblies;
the female contact assemblies comprise a first communication contact; and
the female contact assemblies comprise a second communication contact to provide redundancy relative to the first communication contact.

9. The mechanical circulatory assist system of claim 7, wherein:
the implantable female electrical connector comprises a fluid pathway for venting fluid displaced from the elongated receptacle via insertion of the external male electrical connector into the elongated receptacle; and
the fluid pathway is configured to output the fluid external to the patient.

10. The mechanical circulatory assist system of claim 7, wherein the female connector body comprises radially distributed recesses shaped to interface with a tissue of the patient to inhibit twisting and axial displacement of the implantable female electrical connector relative to the tissue of the patient.

11. The mechanical circulatory assist system of claim 7, wherein the female connector body comprises suture apertures for anchoring of the implantable female electrical connector within the patient.

12. The mechanical circulatory assist system of claim 7, wherein the female connector body is configured for tissue ingrowth for anchoring of the implantable female electrical connector within the patient.

13. The mechanical circulatory assist system of claim 7, wherein:
the implantable female electrical connector comprises wiper seal assemblies;
each of the female contact assemblies is disposed between two of the wiper seal assemblies;
each of the wiper seal assemblies comprises an annular housing and an annular seal supported by the annular housing;
the annular seal is configured to sealing engage the external male electrical connector; and
the wiper seal assembly is configured to cooperate with the external male electrical connector to block passage of fluid along the external male electrical connector.

14. A mechanical circulatory assist system comprising:
a ventricular assist device configured for pumping blood from a ventricle of a patient to an artery of the patient;
an implantable battery configured for supplying power for operation of the ventricular assist device; and
an electrical connector assembly comprising an implantable subdermal electrical connector and an external hypodermic electrical connector, wherein the implantable subdermal electrical connector is configured for receiving electrical power from the external hypodermic electrical connector for recharging of the implantable battery, wherein the implantable subdermal electrical connector is configured to be implanted within the patient under a region of skin of the patient, and wherein the external hypodermic electrical connector is configured to be penetrated through the region of skin of the patient to connect the external hypodermic electrical connector to the implantable subdermal electrical connector.

15. The mechanical circulatory assist system of claim 14, wherein:
the implantable subdermal electrical connector comprises subdermal electrical connector contacts;
a first pair of the subdermal electrical connector contacts is configured for receiving electrical power for recharging of the implantable battery;
a second pair of the subdermal electrical connector contacts is configured for receiving electrical power for recharging of the implantable battery to provide redundancy relative to the first pair of the subdermal electrical connector contacts;
the subdermal electrical connector contacts comprise a first communication contact; and
the subdermal electrical connector contacts comprise a second communication contact to provide redundancy relative to the first communication contact.

16. The mechanical circulatory assist system of claim 14, wherein:
the external hypodermic electrical connector comprises an elongated electrical contact support member and annular electrical contacts mounted to and spaced apart along the elongated electrical contact support member; and
the implantable subdermal electrical connector comprises a subdermal connector body having an elongated receptacle and female contact assemblies disposed in and distributed along the elongated receptacle for interfacing with the annular electrical contacts of the external hypodermic electrical connector.

17. The mechanical circulatory assist system of claim 16, wherein:
the implantable subdermal electrical connector comprises wiper seal assemblies;
each of the female contact assemblies is disposed between two of the wiper seal assemblies; and
each of the wiper seal assemblies comprises an annular housing and an annular seal supported by the annular housing, wherein the annular seal is configured to sealing engage the external hypodermic electrical connector, and the wiper seal assembly is configured to cooperate with the external hypodermic electrical connector to block passage of fluid along the external hypodermic electrical connector.

18. The mechanical circulatory assist system of claim 16, wherein the subdermal connector body comprises radially distributed recesses shaped to interface with a tissue of the patient to inhibit twisting and axial displacement of the implantable subdermal electrical connector relative to the tissue of the patient.

19. The mechanical circulatory assist system of claim 16, wherein the subdermal connector body comprises suture apertures for anchoring of the implantable subdermal electrical connector within the patient.

20. The mechanical circulatory assist system of claim 16, wherein the subdermal connector body is configured for tissue ingrowth for anchoring of the implantable subdermal electrical connector within the patient.
